(19) United States
(12) Patent Application Publication
(10) Pub. No.: US 2024/0091257 A1
(43) Pub. Date: Mar. 21, 2024

(54) USE OF ACTIVATED T-CELLS TO ENHANCE ANTI-TUMOR ACTIVITY OF BISPECIFIC ANTIBODIES OR BITES

(71) Applicants: The Johns Hopkins University, Baltimore, MD (US); Memorial Sloan Kettering Cancer Center, New York, NY (US)

(72) Inventors: Ivan M. Borrello, Baltmore, MD (US); Amy Thomas, Baltimore, MD (US); Eric L. Smith, New York, NY (US)

(21) Appl. No.: 18/039,660

(22) PCT Filed: Dec. 10, 2021
(86) PCT No.: PCT/US2021/062866

§ 371 Date: May 31, 2023

Related U.S. Application Data
(60) Provisional application No. 63/123,913, filed on Dec. 10, 2020.

(51) Int. Cl. A61K 35/17; C07K 16/28; C12N 5/0783

(52) U.S. Cl. CPC A61K 35/17; C07K 16/2809; C07K 16/2818; C07K 16/2878; C12N 5/0636; C07K 2317/31; C12N 2501/51; C12N 2501/515

(57) ABSTRACT

Provided herein are methods of treatment including methods of treating cancer and methods of enhancing an anti-tumoral activity of a bispecific T-cell engager (BiTE) or of a bispecific antibody. The methods disclosed herein include activating T-cells and contacting activated T-cells with BiTEs or bispecific antibodies for administration to a subject.

### CROSS-REFERENCE TO RELATED APPLICATIONS

This application claims the benefit of priority under 35 U.S.C. § 119(e) of U.S. Provisional Patent Application Ser. No. 63/123,913 filed Dec. 10, 2020. The disclosure of the prior application is considered part of and is incorporated by reference in the disclosure of this application.

### BACKGROUND OF THE INVENTION

### Field of the Invention

The present invention relates generally to bispecific T-cell engagers (BiTEs) and bispecific antibodies targeting both tumor antigens and T-cells, and more specifically to the use of activated T-cells to enhance BiTEs and bispecific antibodies anti-tumor activity.

### Background Information

T lymphocytes have the ability to engage in close proximity to tumor cells and as such can induce anti-tumoral T-cell mediated cytotoxicity. However, tumor cells have the ability to induce tumor-specific T-cell tolerance which significantly limits tumor-mediated immune responses. The endogenous T-cell repertoire in patients with a significant tumor burden is intrinsically tolerant towards tumor antigens which leads to profoundly dysfunctional T-cells in cancer-bearing patients.

Bispecific T-cell engagers (BiTEs) and bispecific antibodies targeting both tumor-specific antigen and T-cells have shown significantly greater anti-tumor activity compared to simple antibodies. The reason for this enhanced anti-tumor activity is mediated by the ability of the antibody to bind T-cells in proximity of the tumor and thus increase the tumor-specific T-cell mediated cytotoxicity. BiTEs and bispecific antibodies are currently either FDA approved or being developed in several hematologic and solid tumors. However, the clinical efficacy of BiTEs and bispecific antibodies is dependent on the ability of T-cells to impart anti-tumor activity.

Therefore, there is an unmet need for strategies that enhance T-cell function, which could potentially increase efficacy and anti-tumor efficacy of BiTEs and bispecific antibodies, resulting in enhanced clinical efficacy of BiTEs or bispecific antibodies.

### SUMMARY OF THE INVENTION

The present invention is based on the seminal discovery that the activation of T-cells enhances the anti-tumoral activity of bispecific T-cell engagers (BiTEs) and bispecific antibodies.

In one embodiment, the invention provides a method of treating cancer in a subject including activating T-cells ex vivo and administering to the subject the activated T-cells and a bispecific T-cell engager (BiTE) or a bispecific antibody, wherein the BiTE or the bispecific antibody binds to the activated T-cells and a cancer cell antigen.

In one aspect, the activated T-cell is an activated marrow infiltrating lymphocyte (aMIL) or an activated peripheral blood lymphocyte (aPBL). In some aspects, activating a T-cell includes co-stimulating CD3 and CD28. In various aspects, co-stimulating CD3 and CD28 includes contacting the T-cell with an anti-CD3 antibody and an anti-CD28 antibody. In other aspects, activating a T-cell includes enhancing anti-tumor activity, restoring tumor-mediated immunity and/or reversing T-cell tolerance to tumor antigen. In some aspects, activating a T-cell includes increasing T-cell-mediated tumor cell lysis activity of the T-cell.

In one aspect, the bispecific antibody binds to an antigen. In some aspects, the antigen is a tumor antigen. In one aspect, the tumor antigen is B-cell maturation antigen (BCMA). In another aspect, the bispecific antibody binds to CD3. In one aspect, the activated T-cell is contacted ex vivo with the BiTE or with the bispecific antibody prior to administration to the subject. In another aspect the activated T-cell is infused together with the BiTE or bispecific antibody. In one aspect, the activated T-cell has increased expression of OX40 and/or 4-IBB. In one aspect, the subject has a hematologic cancer or a solid tumor. In some aspects, the subject has leukemia, lymphoma or multiple myeloma. In other aspects, the tumor is selected from prostate, pancreatic, biliary, colon, rectal, liver, kidney, lung, testicular, breast, ovarian, brain, glioblastoma, head and neck cancer, melanoma or sarcoma. In one aspect, an anti-cancer treatment is further administered to the subject. In some aspects, the anti-cancer treatment is selected from the group consisting of surgery, radiotherapy, chemotherapy, immunotherapy, checkpoint inhibitor therapy, and a combination thereof.

In another embodiment, the invention provides a method of enhancing anti-tumor activity of a bispecific T-cell engager (BiTE) or bispecific antibody including activating T-cells and contacting the BiTE or bispecific antibody with the activated T-cell, wherein the BiTE or the bispecific antibody binds to the activated T-cell and a cancer antigen. In one aspect, the activated T-cell has restored T-cell functionality. In other aspects, the T-cell has increased T-cell mediated cytotoxicity. In one aspect, enhancing anti-tumor activity includes restoring tumor-mediated immunity and/or reversing T-cell tolerance to cancer cell antigens. In some aspects, activating the T-cell comprises co-stimulating CD3 and CD28. In various aspects, co-stimulating CD3 and CD28 comprises contacting the T-cell with an anti-CD3 antibody and an anti-CD28 antibody. In one aspect, the tumor is selected from prostate, pancreatic, biliary, colon, rectal, liver, kidney, lung, testicular, breast, ovarian, brain, glioblastoma, head and neck cancer, melanoma or sarcoma.

### BRIEF DESCRIPTION OF THE DRAWINGS

FIG. 1 illustrates contour plots graphs showing the relative expression of OX40 and 4-1BB in activated MILs as compared to activated PBLs when added to myeloma cells in the presence of a bispecific antibody and demonstrates the increased co-expression of OX-40 and 4-1BB of activated MILs following co-incubation with myeloma and the BCMA bispecific antibody.

FIG. 2 is a graph illustrating the percentage of myeloma cell lysis by activated and unactivated PBLs and MILs when combined with the BCMA-specific antibody.

FIGS. 3A-3B shows results of a lysis rechallenge at various effector to target ratios over the various days as indicate. FIG. 3A shows results of a lysis rechallenge with a 1:3 effector to target ratio. FIG. 3B shows results of a lysis rechallenge with a 1:10 effector to target ratio comparing lysis with activated PBLs to activated MILs.

FIG. 4 shows activated MILs enhance the myeloma killing of bispecific antibodies.

FIGS. 5A-5C shows graphs illustrating T cell activation enhances bispecific-mediated tumor killing. FIG. 5A shows cell killing as determined by percent lysis determined by co-culturing a BCMA-specific bispecific antibody with either activated or unactivated PBLs or MILs as indicated in H929 (a high BCMA expressing line) and OPM-2 (low BCMA expressing) or the OPM-2 BCMA knock-out line. FIG. 5B shows T cell activation determined by 4-1BB and OX40 expression determined by co-culturing a BCMA-specific bispecific antibody with either activated or unactivated PBLs or MILs as indicated in H929 (a high BCMA expressing line) and OPM-2 (low BCMA expressing) or the OPM-2 BCMA knock-out line. FIG. 5C shows polyfunctionality via TNF-alpha and interferon-gamma expression determined by co-culturing a BCMA-specific bispecific antibody with either activated or unactivated PBLs or MILs as indicated in H929 (a high BCMA expressing line) and OPM-2 (low BCMA expressing) or the OPM-2 BCMA knock-out line.

FIGS. 6A-6I shows graphs illustrating activated MILs show superior cytotoxicity compared to PBLs when combined with a bispecific antibody. FIG. 6A shows cell killing as determined by percent lysis determined by co-culturing a BCMA-specific bispecific antibody with activated PBLs or activated MILs as indicated in 3817/3818 MM cells. FIG. 6B shows CD3 fold expansion determined by co-culturing a BCMA-specific bispecific antibody with activated PBLs or activated MILs as indicated in 3817/3818 MM cells. FIG. 6C shows T cell exhaustion as determined by the expression of PD-1 and TIGIT determined by co-culturing a BCMA-specific bispecific antibody with activated PBLs or activated MILs as indicated in 3817/3818 MM cells. FIG. 6D shows cell killing as determined by percent lysis determined by co-culturing a BCMA-specific bispecific antibody with activated PBLs or activated MILs as indicated in 4238/4239 MM cells. FIG. 6E shows CD3 fold expansion determined by co-culturing a BCMA-specific bispecific antibody with activated PBLs or activated MILs as indicated in 4238/4239 MM cells. FIG. 6F shows T cell exhaustion as determined by the expression of PD-1 and TIGIT determined by co-culturing a BCMA-specific bispecific antibody with activated PBLs or activated MILs as indicated in 4238/4239 MM cells. FIG. 6G shows cell killing as determined by percent lysis determined by co-culturing a BCMA-specific bispecific antibody with activated PBLs or activated MILs as indicated in 4556/4557 MM cells. FIG. 6H shows CD3 fold expansion determined by co-culturing a BCMA-specific bispecific antibody with activated PBLs or activated MILs as indicated in 4556/4557 MM cells. FIG. 6I shows T cell exhaustion as determined by the expression of PD-1 and TIGIT determined by co-culturing a BCMA-specific bispecific antibody with activated PBLs or activated MILs as indicated in 4556/4557 MM cells.

FIGS. 7A-7C shows plots of CD107a and intracellular cytokine staining of CD3+ aPBL or aMIL after 48 hour coculture with OPM-2 (1:10) E:T ratio with 10⁻⁸ M BCMA-specific bispecific antibody. FIG. 7A shows CD107a staining. FIG. 7B shows IGNg staining. FIG. 7C shows TNFa staining.

### DETAILED DESCRIPTION OF THE INVENTION

The present invention is based on the seminal discovery that the activation of T-cell enhances the anti-tumoral activity of bispecific T-cell engagers (BiTEs) and bispecific antibodies.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The methods and materials are now described herein.

In one embodiment, the invention provides a method of treating cancer in a subject including activating a T-cell, and administering to the subject the activated T-cell, and a bispecific T-cell engager (BiTE) or a bispecific antibody, wherein the BiTE or the bispecific antibody binds to the activated T-cell and a cancer cell antigen.

The term “cancer” refers to a group of diseases characterized by abnormal and uncontrolled cell proliferation starting at one site (primary site) with the potential to invade and to spread to other sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof.

As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

The methods described herein are for the treatment of cancer in a subject. The “subject”, which refers to any individual or patient to which the subject methods are performed is generally human, although as will be appreciated by those in the art, the subject may be an animal, for example, a mammal. Thus, the subject can be a human or veterinary patient, under the treatment of a clinician, e.g., physician. The term “subject” generally refers to the individual who is the target of administration or treatment.

The term “treatment” is used interchangeably herein with the term “therapeutic method” and refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of cancer). The effective amount can be determined as described herein. A therapeutically effective dose refers to an amount of the composition that is sufficient to ameliorate one or more causes or symptoms of a cancer. Such amelioration only requires a reduction or alteration, not necessarily elimination.

The terms “administration of” and or “administering” should be understood to mean providing a pharmaceutical composition in a therapeutically effective amount to the subject in need of treatment. Administration routes can be enteral or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization.

The methods described herein for the treatment of cancer rely on the administration of a bispecific antibody or a bispecific T-cell engager (BiTE), and an activated T-cell, wherein the BiTE or the bispecific antibody binds to the activated T-cell and a cancer cell antigen.

As used herein, the term “antibody,” refers to any polypeptide comprising an antigen-binding site regardless of the source, species of origin, method of production, and characteristics.

The term “antibody,” can refer to immunoglobulin molecules and immunologically active portions of immunoglobulin molecules, i.e., molecules that contain an antigen binding site that immunospecifically binds an antigen. “Native antibodies” and “intact immunoglobulins”, or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known.

Antibodies include natural or artificial, mono- or polyvalent antibodies including, but not limited to, polyclonal, monoclonal, multispecific, human, humanized or chimeric antibodies, single chain antibodies, and antibody fragments. The term “antibody fragment” refers to any derivative of an antibody which is less than full-length. Antibody fragments include a portion of an intact antibody, and can include the antigen binding or variable region of the intact antibody. Examples of antibody fragments include Fab, Fab′ and F(ab′)2, Fc fragments or Fc-fusion products, single-chain Fvs (scFv), disulfide-linked Fvs (sdfv) and fragments including either a VL or VH domain; diabodies, tribodies and the like (Zapata et al. Protein Eng. 8(10):1057-1062 [1995]).

The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “binding specificity” of a bispecific antibody refers to its “antigen-binding domains”, the parts of the bispecific antibody molecule that comprise the area that specifically binds to or complements to a part or all of an antigen. Where an antigen is large, an antibody may only bind to a particular part of the antigen. The “epitope” or “antigenic determinant” is a portion of an antigen molecule that is responsible for interactions with the antigen-binding domain of an antibody. An antigen-binding domain may be provided by one or more antibody variable domains (e.g., a so-called Fd antibody fragment consisting of a VH domain). An antigen-binding domain may comprise an antibody light chain variable region (VL) and an antibody heavy chain variable region (VH).

The bispecific antibodies, or BiTEs described herein have two antigen binding domains, and therefore can recognize and bind to two antigens. An “antigen” according to the invention covers any substance that will elicit an immune response. In particular, an “antigen” relates to any substance, such as a peptide or protein, that reacts specifically with antibodies or T-lymphocytes. The term “antigen” can refer to any molecule which comprises at least one epitope. In some embodiments, an antigen in the context of the present invention is a molecule which, optionally after processing, induces an immune reaction. According to the present invention, any suitable antigen may be used, which is a candidate for an immune reaction, wherein the immune reaction includes a cellular immune reaction. In the context of the embodiments of the present invention, the antigen can be presented by a cell, such as by an antigen presenting cell which includes a diseased cell, in particular a cancer cell, in the context of MHC molecules, which results in an immune reaction against the antigen. An antigen is a product which corresponds to or is derived from a naturally occurring antigen. Such naturally occurring antigens include tumor antigens. The term “epitope” refers to an antigenic determinant in a molecule such as an antigen, i.e., to a part in or fragment of the molecule that is recognized by the immune system. An epitope of a protein such as a tumor antigen is a continuous or discontinuous portion of said protein.

A “bispecific antibody” refers to an antibody having two different antigen-binding regions defined by different antibody sequences. This can be understood as different target binding but includes binding to different epitopes in one target. Bispecific antibodies can be prepared as full-length antibodies or antibody fragments (e.g., F(ab′)2 bispecific antibodies). Bispecific antibodies may contain a heavy chain comprising one or more variable regions and/or a light chain comprising one or more variable regions. Bispecific antibodies can be constructed using only antibody variable domains. A fairly efficient and relatively simple method is to make the linker sequence between the VH and VL domains so short that they cannot fold over and bind one another. Reduction of the linker length to 3-12 residues prevents the monomeric configuration of the scFv molecule and favors intermolecular VH-VL pairings with formation of a 60 kDa non-covalent scFv dimer “diabody”. The diabody format can also be used for generation of recombinant bispecific antibodies, which are obtained by the noncovalent association of two single-chain fusion products, consisting of the VH domain from one antibody connected by a short linker to the VL domain of another antibody. Reducing the linker length still further below three residues can result in the formation of trimers (“triabody”, about 90 kDa) or tetramers (“tetrabody”, about 120 kDa). For a review of engineered antibodies, particularly single domain fragments, see Holliger and Hudson, 2005, Nature Biotechnology, 23: 1126-1 136.

Methods for making bispecific antibodies are known in the art. Traditional production of full-length bispecific antibodies is based on the co-expression of two immunoglobulin heavy chain-light chain pairs, where the two chains have different specificities (Millstein et al., Nature, 305:537-539 [1983]). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

Bispecific T-cell engagers or BiTEs® are a class of artificial bispecific monoclonal antibodies that are investigated for the use as anti-cancer drugs. They direct a host's immune system, more specifically the T-cells' cytotoxic activity, against cancer cells. BiTEs® are fusion proteins consisting of two single-chain variable fragments (scFvs) of different antibodies, or amino acid sequences from four different genes, on a single peptide chain of about 55 kilodaltons. One of the scFvs binds to T-cells via the CD3 receptor, and the other to a tumor cell via a tumor-specific molecule.

As used herein, “fusion protein” or “fusion polypeptide” refers to a hybrid polypeptide which comprises polypeptide portions from at least two different polypeptides. The portions may be from proteins of the same organism, in which case the fusion protein is said to be “intraspecies”, “intragenic”, etc. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. A first polypeptide may be fused to the N-terminus, the C-terminus, or the N- and C-terminus of a second polypeptide. Furthermore, a first polypeptide may be inserted within the sequence of a second polypeptide.

Like other bispecific antibodies, and unlike ordinary monoclonal antibodies, BiTEs® form a link between T-cells and tumor cells. This causes T-cells to exert cytotoxic activity on tumor cells by producing proteins like perforin and granzymes, independently of the presence of MHC I or co-stimulatory molecules. These proteins enter tumor cells and initiate the cell's apoptosis. This action mimics the physiological processes observed during T-cell attacks against tumor cells. Examples of BiTEs® include blinatumomab, which links T-cells with CD19 receptors found the surface of B-cells, and which is approved for the treatment of adults with Philadelphia chromosome-negative relapsed or refractory acute lymphoblastic leukemia; and solitomab links T-cells with the EpCAM antigen which is expressed by colon, gastric, prostate, ovarian, lung, and pancreatic cancers.

The BiTEs® and bispecific antibodies described herein have a binding activity against two different targets, with one of the targets being an antigen present on a T-cell, such as an immune cell receptor, (e.g., the CD3 receptor present on killer T immune cells), the second one being a tumor antigen. The concept is that the BITE antibody will activate the killer T-cell once it binds to the T cell antigen, while the other end of the antibody binds the tumor antigen on the surface of the cancer cell. The activated T-cell will be held in close proximity to the cancer cell and start to kill the cancer cell through an immune mediated attack.

T-cells, T-lymphocytes or T-cells are a type of lymphocyte, which develop in the thymus gland and play a central role in the immune response. T cells can be distinguished from other lymphocytes by the presence of a T-cell receptor (TCR) on the cell surface. These immune cells originate as precursor cells, derived from bone marrow, and develop into several distinct types of T cells once they have migrated to the thymus gland. T cell differentiation continues even after they have left the thymus. T-cells play a central role in cell-mediated immunity. There are two major subtypes of T-cells: the killer T-cell and the helper T-cell. In addition, there are suppressor T-cells which have a role in modulating immune response. Killer T-cells only recognize antigens coupled to Class I MHC molecules, while helper T cells only recognize antigens coupled to Class II MHC molecules. These two mechanisms of antigen presentation reflect the different roles of the two types of T-cell. A third minor subtype are the γδ T cells that recognize intact antigens that are not bound to MHC receptors.

T-cells mediate “cellular immune response”, “cellular response”, or “cellular response against an antigen”, which, along with any additional similar terms refers to cellular response directed to cells characterized by presentation of an antigen with class I or class II MHC. The helper T-cells (also termed CD4+ T-cells) play a central role by regulating the immune response and the killer cells (also termed cytotoxic T-cells, cytolytic T-cells, CD8+ T-cells or CTLs) kill diseased cells such as cancer cells, preventing the production of more diseased cells. In some embodiments, the present invention involves the enhancement of an anti-tumor CTL response against tumor cells expressing one or more tumor-specific antigens and includes presenting such tumor expressed antigens with class I MHC.

Groups of specific, differentiated T-cells have an important role in controlling and shaping the immune response by providing a variety of immune-related functions. One of these functions is immune-mediated cell death, and it is carried out by T cells in several ways: CD8+ T-cells, also known as “killer cells”, are cytotoxic—this means that they are able to directly kill virus-infected cells as well as cancer cells. CD8+ T-cells are also able to utilize small signaling proteins, known as cytokines, to recruit other cells when mounting an immune response. A different population of T cells, the CD4+ T-cells, function as “helper cells”. Unlike CD8+ killer T-cells, these CD4+ helper T-cells function by indirectly killing cells identified as foreign: they determine if and how other parts of the immune system respond to a specific, perceived threat. Helper T-cells also use cytokine signaling to influence regulatory B cells directly, and other cell populations indirectly. Regulatory T cells are yet another distinct population of these cells that provide the critical mechanism of tolerance, whereby immune cells are able to distinguish invading cells from “self”—thus preventing immune cells from inappropriately mounting a response against oneself (which would by definition be an “autoimmune” response). For this reason, these regulatory T-cells have also been called “suppressor” T-cells. These same self-tolerant cells are co-opted by cancer cells to prevent the recognition of, and an immune response against, tumor cells.

All T-cells originate from c-kit+Scal+ hematopoietic stem cells (HSC) which reside in the bone marrow. The HSC then differentiate into multipotent progenitors (MPP) which retain the potential to become both myeloid and lymphoid cells. The process of differentiation then proceeds to a common lymphoid progenitor (CLP), which can only differentiate into T, B or NK cells. These CLP cells then migrate via the blood to the thymus, where they engraft. The earliest cells which arrived in the thymus are termed double-negative, as they express neither the CD4 nor CD8 co-receptor. The newly arrived CLP cells are CD4-CD8-CD44+CD25-ckit+ cells and are termed early thymic progenitors (ETP) cells. These cells will then undergo a round of division and downregulate c-kit and are termed DN1 cells. Upon encounter with an antigen-presenting cell (APC) such as a dendritic cell or a B-cell displaying antigen fragments bound to its MHC molecules, the simultaneous engagement of T-cell receptor (such as the CD3) and a co-stimulatory molecule (such as CD28 or ISOC) induces the activation of the T-cell, which is required for the production of an effective immune response.

The activated T-cells described herein can be inactivated T-cells collected from a patient, activated ex vivo and administered to the patient. For example, the T-cells can be obtained from the bone marrow (marrow infiltrating lymphocytes, MILs), or from the blood (peripheral blood lymphocytes, PBL). Any other source of T-cells can be used from a subject, activating the cells ex vivo, and administering them back to the subject after contacting the cells with the bispecific antibody or BiTE.

Marrow infiltrating lymphocytes or MTLs refers to a preparation of cells, which consists of autologous marrow infiltrating lymphocytes (MILs), that can be manipulated in vitro, and which present potential antitumor and immune stimulating activities. Peripheral blood lymphocytes (PBLs) are mature lymphocytes that circulate in the blood, rather than localizing to organs (such as the spleen or lymph nodes). They comprise T cells, NK cells and B cells.

MTLs and PBLs can be harvested from autologous patients, and manipulated ex vivo, for example to activate T-cells. T-cell activation can be performed by the co-stimulation of CD3 and CD28, for example by contacting the T-cell with an anti-CD3 antibody and an anti-CD28 antibody. The cells can be exposed to and activated by anti-CD3/anti-CD28 monoclonal antibodies covalently attached to super-paramagnetic microbeads. After removal of the beads and expansion of the cells in culture, the activated MILs (aMTLs) or PBLs (aPBLs) are re-introduced into the patient. aMTLs and aPBLs can then infiltrate the tumor microenvironment and initiate tumor cell lysis. The method used for the activation of the T-cells, including the reagents, or the process itself is not limiting to the practice of the described methods. Any T-cell activation process resulting in the generation of cytotoxic T-cells capable of tumor-lysis activity can be used for the activation of the T-cells.

There are several ways to evaluate T-cell activation. For example, the expression of one or more proteins at the surface of the T-cell can be indicative of T-cell activation.

In one aspect, the activated T-cell when combined with the bispecific has increased expression of OX40 and/or 4-IBB suggestive of significant activation.

Tumor necrosis factor receptor superfamily, member 4 (TNFRSF4), also known as CD134 and OX40 receptor, is a member of the TNFR-superfamily of receptors which is not constitutively expressed on resting naïve T cells, unlike CD28. OX40 is a secondary co-stimulatory immune checkpoint molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels. OX40L binds to OX40 receptors on T-cells, preventing them from dying and subsequently increasing cytokine production. OX40 has a critical role in the maintenance of an immune response beyond the first few days and onwards to a memory response due to its ability to enhance survival. OX40 also plays a crucial role in both Th1 and Th2 mediated reactions in vivo.

CD137 is a member of the tumor necrosis factor (TNF) receptor family, also known as tumor necrosis factor receptor superfamily member 9 (TNFRSF9), 4-1BB and induced by lymphocyte activation (ILA). 4-1BB is a co-stimulatory immune checkpoint molecule expressed by activated T-cells of both the CD4+ and CD8+ lineages. Although it is thought to function mainly in co-stimulating those cell types to support their activation by antigen presenting cells expressing its ligand (CD137L), 4-1BB is also expressed on dendritic cells, B cells, NK cells, neutrophils and macrophages. The best characterized activity of 4-1BB is its costimulatory activity for activated T-cells. Crosslinking of CD137 enhances T-cell proliferation, IL-2 secretion, survival and cytolytic activity. Further, it can enhance immune activity to eliminate tumors in mice.

Alternatively, or complementarily, the production of cytokines produced by activated T-cells can be indicative of T-cell activation. For example, the production of IL-2 and/or IFNγ; the detection of cytotoxic T-cell proliferation; or the expression of cytotoxic T-cell receptor CD8 can be an indication of T-cells activation.

In some aspects, activating a T-cell includes enhancing anti-tumor activity, restoring tumor-mediated immunity and/or reversing T-cell tolerance to tumor antigen. In other aspects, activating a T-cell includes increasing T-cell-mediated tumor cell lysis activity of the T-cell.

Upon activation of a T-cell through, for example, the co-stimulation of CD3 and CD28, T-cells start secreting cytokines. Some cytokines induce the proliferation of larger amount of T-cells, some cytokines induce the differentiation of T-cells into cytotoxic cells that track down target cells (such as cancer cells), and other cytokines induce the differentiation of T-cells into helper cells, that secrete cytokines to attract macrophages, neutrophils and other lymphocytes to the site. Proliferation and differentiation of T-cells includes the acquisition of the cell functionality of the T-cell. For example, this can include the differentiation of T-cells into cytotoxic T-cells, and the induction of T-cell effector function including target cells (cancer cells) lysis through the release of cytotoxins perforin and granzymes. By targeting cancer-cells with the BiTE® or bispecific antibody, T-cell activation enhances tumor-specific cytotoxicity, and therefore anti-tumoral activity. The enhancement of anti-tumoral activity can in turn result in the restoration of tumor-mediated immunity (i.e., the recognition of tumor cells by immune cells), and therefore in the reversing of T-cell tolerance to tumor antigen (i.e., the non-recognition of tumor antigen as “foreign”).

The bispecific antibody described herein can bind to a tumor antigen and a T-cell.

As used herein, the term “tumor antigen” refers to an antigenic protein, polypeptide or fragment thereof produced in tumor cells, and that triggers an immune response in the host. Tumor antigens are useful tumor markers in identifying tumor cells with diagnostic tests and are potential candidates for use in cancer therapy. Normal proteins in the body are not antigenic because of self-tolerance, a process in which self-reacting cytotoxic T lymphocytes (CTLs) and autoantibody-producing B lymphocytes are discarded “centrally” in primary lymphatic tissue (BM) and “peripherally” in secondary lymphatic tissue (mostly thymus for T-cells and spleen/lymph nodes for B cells). Thus, any protein that is not exposed to the immune system triggers an immune response. This may include normal proteins that are well sequestered from the immune system, proteins that are normally produced in extremely small quantities, proteins that are normally produced only in certain stages of development, or proteins whose structure is modified due to mutation.

Tumor antigen can be classified into two categories based on their pattern of expression: tumor-specific antigens (TSA), which are present only on tumor cells and not on any other cell and tumor-associated antigens (TAA), which are present on some tumor cells and also some normal cells. Any TSA or TAA can be used and combined with a T-cell antigen for the generation of the bispecific antibodies or BiTEs described herein. Virtually all TSA and TAA can be used as a tumor antigen.

The bispecific antibody can bind to any tumor antigen. For example, the tumor antigen can a B-cell maturation antigen. B-cell maturation antigen (BCMA or BCM), also known as tumor necrosis factor receptor superfamily member 17 (TNFRSF17), is a protein that in humans is encoded by the TNFRSF17 gene. TNFRSF17 is a cell surface receptor of the TNF receptor superfamily which recognizes B-cell activating factor (BAFF). The protein is preferentially expressed in mature B lymphocytes and may be important for B cell development and autoimmune response. This receptor has been shown to specifically bind to the tumor necrosis factor (ligand) superfamily, member 13b (TNFSF13B/TALL-1/BAFF), and to lead to NF-κB and MAPK8/JNK activation. This receptor also binds to various TRAF family members, and thus may transduce signals for cell survival and proliferation. Overexpression, or abnormal expression of BCMA have been observed in hematological cancer, linking BCMA to leukemia, lymphomas, and multiple myeloma, in which the expression of the protein is considered a tumor antigen.

The T-cell antigen can be any of the protein or receptor expressed at the surface of T-cell. For example, the T-cell antigen can be CD3 a protein complex and T cell co-receptor that is involved in activating both the cytotoxic T cell and T helper cells. It is composed of four distinct chains. In mammals, the complex contains a CD3γ chain, a CD3δ chain, and two CD3ε chains.

In one aspect, the bispecific antibody binds to B-cell maturation antigen (BCMA). In another aspect, the bispecific antibody binds to CD3.

In one aspect, the activated T-cell is contacted ex vivo with the BiTE or with the bispecific antibody prior to administration to the subject.

Once the T-cells have been activated (i.e., once the effector functions of the T cells including tumor lysis functionality have been restored or enhanced), the activated T-cells are contacted with the BiTEs or bispecific antibodies, which recognize and bind to the activated T-cells via specific binding of one of the two binding specificities of the antibodies. The activated T-cells are contacted with the BiTE or with the bispecific antibody prior to administration to the subject so that upon administration and specific binding of the BiTE or bispecific antibody to the tumor antigen, the activated T-cell, with enhanced tumor lysis capability and restored anti-tumor immunity can be in close proximity to the cancer cell.

The BiTE or bispecific antibody has two binding specificities, such that it can specifically bind to an activated or unactivated T-cell and a cancer cell, through the specific binding to a tumor antigen of interest. Depending on the tumor antigen, the cancer can be of any type.

The bispecific antibodies described here, in combination with an activated T-cell can used for the treatment of a variety of cancer types.

The most common types of cancer in males are lung cancer, prostate cancer, colorectal cancer and stomach cancer. In females, the most common types are breast cancer, colorectal cancer, lung cancer and cervical cancer. However, there are more 100 cancer types. Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood’, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland’ Cancer, Childhood; Sarcoma, Ewing's Family of Tumors; Sarcoma, Kaposi's; Sarcoma (OsteosarcomaVMalignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

The cancer can, for example initiate in blood-forming tissue, such as the bone marrow, or in the cells of the immune system, and be referred to as hematologic cancer, or blood cancer. Hematologic cancers affect the production and function of blood cells, and are classified in three main types: leukemia, lymphoma, and multiple myeloma. As used herein, “leukemia” refers to a blood caused by the rapid production of abnormal white blood cells. Examples of leukemia include acute lymphoblastic leukemia (ALL), acute myeloid leukemia (AML), chronic lymphocytic leukemia, chronic myelogenous leukemia, myelodysplastic syndromes, myeloproliferative neoplasms and hairy cell leukemia. As used herein, “lymphoma” refers to a type of blood cancer that affects the lymphatic system. Examples of lymphoma include AIDS-related lymphoma, cutaneous T-cell lymphoma, Hodgkin lymphoma, Hodgkin lymphoma, mycosis fungoides, non-Hodgkin lymphoma, primary central nervous system lymphoma, Sezary syndrome, cutaneous T-Cell lymphoma, and Waldenström's macroglobulinemia. As used herein, “myeloma” is a cancer of the plasma cells. Examples of myeloma include smoldering myeloma, active myeloma, plasma cell leukemia, and amyloidosis.

In one aspect, the subject has a hematologic cancer or a solid tumor. In some aspects, the subject has leukemia, lymphoma or multiple myeloma.

As used herein, “leukemia” refers to a blood caused by the rapid production of abnormal white blood cells. Examples of leukemia include acute lymphoblastic leukemia (ALL), acute myeloid leukemia (AML), chronic lymphocytic leukemia, chronic myelogenous leukemia, and hairy cell leukemia.

As used herein, “lymphoma” refers to a type of blood cancer that affects the lymphatic system. Examples of lymphoma include AIDS-related lymphoma, cutaneous T-cell lymphoma, Hodgkin lymphoma, Hodgkin lymphoma, mycosis fungoides, non-Hodgkin lymphoma, primary central nervous system lymphoma, Sezary syndrome, cutaneous T-Cell lymphoma, and Waldenström macroglobulinemia.

As used herein, “myeloma” is a cancer of the plasma cells. Examples of myeloma include smoldering myeloma, active myeloma, plasma cell leukemia, and amyloidosis.

In other aspects, the tumor is selected from prostate, pancreatic, biliary, colon, rectal, liver, kidney, lung, testicular, breast, ovarian, brain, glioblastoma, head and neck cancer, melanoma or sarcoma.

Cancer are complicated diseases, that can be treated using various therapeutic approaches, based on the cancer type, the cancer stage, the patient's general health status (including age, and additional disease or conditions). In many cases, cancers are treated using a combination of therapeutic methods concurrently or consecutively, in an attempt to increase the patient response, and ultimately the patient's survival.

In one aspect, an anti-cancer treatment is further administered to the subject.

In some aspects, the anti-cancer treatment is selected from the group consisting of surgery, radiotherapy, chemotherapy, immunotherapy, checkpoint inhibitor therapy, and a combination thereof.

In some aspects, administration can be in combination with one or more additional therapeutic agents. The phrases “combination therapy”, “combined with” and the like refer to the use of more than one medication or treatment simultaneously to increase the response. The composition of the present invention might for example be used in combination with other drugs or treatment in use to treat cancer. Specifically, the administration of the composition of the present invention to a subject can be in combination with any anti-cancer therapies. Such therapies can be administered prior to, simultaneously with, or following administration of the composition of the present invention.

The term “anti-cancer therapy” or “anti-cancer treatment” as used herein is meant to refer to any treatment that can be used to treat cancer, such as surgery, radiotherapy, chemotherapy, immunotherapy, and checkpoint inhibitor therapy.

Examples of chemotherapy include treatment with a chemotherapeutic, cytotoxic or antineoplastic agents including, but not limited to, (i) anti-microtubules agents comprising vinca alkaloids (vinblastine, vincristine, vinflunine, vindesine, and vinorelbine), taxanes (cabazitaxel, docetaxel, larotaxel, ortataxel, paclitaxel, and tesetaxel), epothilones (ixabepilone), and podophyllotoxin (etoposide and teniposide); (ii) antimetabolite agents comprising anti-folates (aminopterin, methotrexate, pemetrexed, pralatrexate, and raltitrexed), and deoxynucleoside analogues (azacitidine, capecitabine, carmofur, cladribine, clofarabine, cytarabine, decitabine, doxifluridine, floxuridine, fludarabine, fluorouracil, gemcitabine, hydroxycarbamide, mercaptopurine, nelarabine, pentostatin, tegafur, and thioguanine); (iii) topoisomerase inhibitors comprising Topoisomerase I inhibitors (belotecan, camptothecin, cositecan, gimatecan, exatecan, irinotecan, lurtotecan, silatecan, topotecan, and rubitecan) and Topoisomerase II inhibitors (aclarubicin, amrubicin, daunorubicin, doxorubicin, epirubicin, etoposide, idarubicinm, merbarone, mitoxantrone, novobiocin, pirarubicin, teniposide, valrubicin, and zorubicin); (iv) alkylating agents comprising nitrogen mustards (bendamustine, busulfan, chlorambucil, cyclophosphamide, estramustine phosphate, ifosamide, mechlorethamine, melphalan, prednimustine, trofosfamide, and uramustine), nitrosoureas (carmustine (BCNU), fotemustine, lomustine (CCNU), N-Nitroso-N-methylurea (MNU), nimustine, ranimustine semustine (MeCCNU), and streptozotocin), platinum-based (cisplatin, carboplatin, dicycloplatin, nedaplatin, oxaliplatin and satraplatin), aziridines (carboquone, thiotepa, mytomycin, diaziquone (AZQ), triaziquone and triethylenemelamine), alkyl sulfonates (busulfan, mannosulfan, and treosulfan), non-classical alkylating agents (hydrazines, procarbazine, triazenes, hexamethylmelamine, altretamine, mitobronitol, and pipobroman), tetrazines (dacarbazine, mitozolomide and temozolomide); (v) anthracyclines agents comprising doxorubicin and daunorubicin. Derivatives of these compounds include epirubicin and idarubicin; pirarubicin, aclarubicin, and mitoxantrone, bleomycins, mitomycin C, mitoxantrone, and actinomycin; (vi) enzyme inhibitors agents comprising FI inhibitor (Tipifarnib), CDK inhibitors (Abemaciclib, Alvocidib, Palbociclib, Ribociclib, and Seliciclib), PrI inhibitor (Bortezomib, Carfilzomib, and Ixazomib), PhI inhibitor (Anagrelide), TMPDI inhibitor (Tiazofurin), LI inhibitor (Masoprocol), PARP inhibitor (Niraparib, Olaparib, Rucaparib), HDAC inhibitor (Belinostat, Panobinostat, Romidepsin, Vorinostat), and PIKI inhibitor (Idelalisib); (vii) receptor antagonist agent comprising ERA receptor antagonist (Atrasentan), Retinoid X receptor antagonist (Bexarotene), Sex steroid receptor antagonist (Testolactone); (viii) ungrouped agent comprising Amsacrine, Trabectedin, Retinoids (Alitretinoin Tretinoin) Arsenic trioxide, Asparagine depleters (Asparaginase/Pegaspargase), Celecoxib, Demecolcine Elesclomol, Elsamitrucin, Etoglucid, Lonidamine, Lucanthone, Mitoguazone, Mitotane, Oblimersen, Omacetaxine mepesuccinate, and Eribulin.

Examples of immunotherapy include treatment with antibodies including, but not limited to, alemtuzumab, Avastin (bevacizumab), Bexxar (tositumomab), CDP 870, and CEA-Scan (arcitumomab), denosumab, Erbitux (cetuximab), Herceptin (trastuzumab), Humira (adalimumab), IMC-IIF 8, LeukoScan (sulesomab), MabCampath (alemtuzumab), MabThera (Rituximab), matuzumab, Mylotarg (gemtuzumab oxogamicin), natalizumab, NeutroSpec (Technetium (99mTc) fanolesomab), panitumamab, Panorex (Edrecolomab), ProstaScint (Indium-Ill labeled Capromab Pendetide), Raptiva (efalizumab), Remicade (infliximab), ReoPro (abciximab), rituximab, Simulect (basiliximab), Synagis (palivizumab), TheraCIM hR3, tocilizumab, Tysabri (natalizumab), Verluma (nofetumomab), Xolair (omalizumab), Zenapax (dacliximab), Zevalin (ibritumomab tiuxetan (IDEC-Y2B8) conjugated to yttrium 90), Gilotrif (afatinib), Lynparza (olaparib), Perjeta (pertuzumab), Otdivo (nivolumab), Bosulif (bosutinib), Cabometyx (cabozantinib), trastuzumab-dkst (Ogivri), Sutent (sunitinib malate), Adcetris (brentuximab vedotin), Alecensa (alectinib), Calquence (acalabrutinib), Yescarta (ciloleucel), Verzenio (abemaciclib), Keytruda (pembrolizumab), Aliqopa (copanlisib), Nerlynx (neratinib), Imfinzi (durvalumab), Darzalex (daratumumab), Tecentriq (atezolizumab), and Tarceva (erlotinib).

“Checkpoint inhibitor therapy” is a form of cancer treatment that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM-3 (T-cell Immunoglobulin domain and Mucin domain 3), and VISTA (V-domain Ig suppressor of T cell activation).

Immunotherapy also includes the use of adoptive transfer of genetically engineered T cells, modified to recognize and eliminate cancer cells specifically. For example, T cells can be genetically modified to stably express on their surface chimeric antigen receptors (CAR). CAR are synthetic proteins comprising of a signaling endodomain, consisting of an intracellular domain of the CD3-zeta chain, a transmembrane domain, and an extracellular domain consisting of the antigen recognition fragment of a monoclonal antibody which gives the receptor its specificity for tumor associated antigen (e.g. an scFv, or single chain variable region fragment). Upon interaction with the target cancer cell expressing the scFv's cognate antigen, the chimeric antigen receptor triggers an intracellular signaling leading to T-cell activation and to a cytotoxic immune response against tumor cells. Such therapies have been shown to be efficient against relapsed/refractory disease. Additionally, CAR-T cells can be engineered to include co-stimulatory receptor that enhance the T-cell-mediated cytotoxic activity. Furthermore, CAR-T cells can be engineered to produce and deliver protein or an agent of interest in the tumor microenvironment.

In addition, activated MILs, as exemplified in the Examples herein, can be used to enhance anti-tumor activity of bispecific antibodies or BiTEs.

In another embodiment, the invention provides method of enhancing anti-tumor activity of a bispecific T-cell engager (BiTE) or bispecific antibody including activating a T-cell, and contacting the BiTE or bispecific antibody with the activated T-cell, wherein the BiTE or the bispecific antibody binds to the activated T-cell and a cancer antigen.

In one aspect, the activated T-cell has restored T-cell functionality. In other aspects, the T-cell has increased T-cell mediated cytotoxicity.

In one aspect, enhancing anti-tumor activity includes restoring tumor-mediated immunity and/or reversing T-cell tolerance to cancer cell antigen. In some aspects, activating the T-cell comprises co-stimulating CD3 and CD28. In various aspects, co-stimulating CD3 and CD28 comprises contacting the T-cell with an anti-CD3 antibody and an anti-CD28 antibody. In one aspect, the tumor is selected from prostate, pancreatic, biliary, colon, rectal, liver, kidney, lung, testicular, breast, ovarian, brain, glioblastoma, head and neck cancer, melanoma or sarcoma.

Presented below are examples the combination of activated T-cell and discussing bispecific T-cell engager and bispecific antibody that bind to the activated T-cell and a cancer cell antigen, contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Utilizing Activated MILs to Enhance Anti-Tumor Activity of Bispecific Antibodies or BiTEs

To assess the impact of T-cells activation on T-cell activity and tumor-antigen T-cell tolerance, T-lymphocytes were activated, and bispecific antibody efficacy was evaluated.

In light of previous demonstration of the superior anti-tumor efficacy of MILs as compared to PBLs, reverse tolerance and T-cell functionality restoration were examined using both activated MILs and PBLs, and the results were compared to those obtained using non-activated MILs and PBLs.

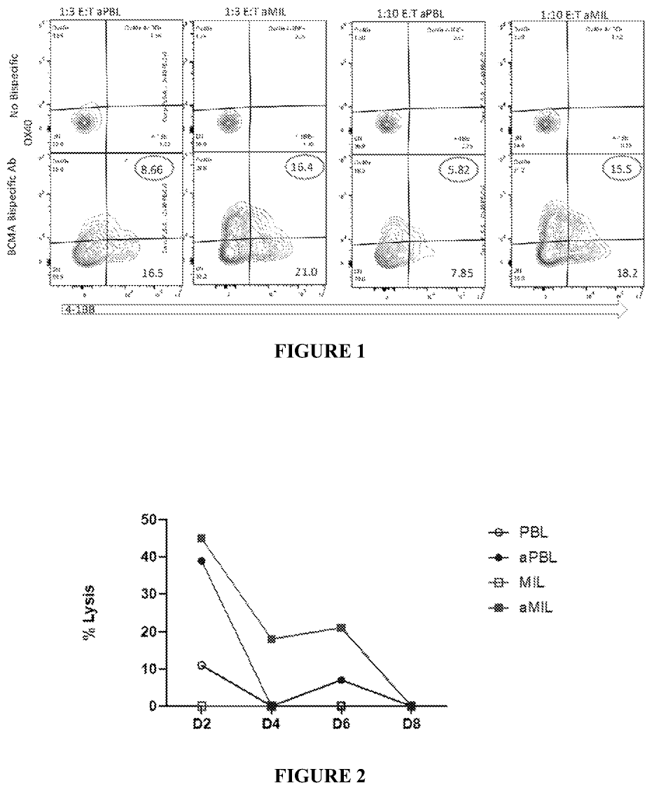

T lymphocytes in MILs and PBLs were activated, and activation was evaluated by flow cytometry by assessing the level of expression of OX40 and 4-1BB in the cells. As illustrated in FIG. 1, it was found that the activation of MILs demonstrated greater activation compared to activated PBLs in terms of expression of OX40 and 4-1BB. Indeed, co-culturing of T cells with the BCMA bispecific antibody showed significantly greater expression and co-expression of OX-40 and 4-1BB of activated MILs as compared to activated PBLs.

To fully model the various scenarios, tumor killing experiments were performed. A BCMA bispecific antibody was co-cultured with either inactivated T-cells from myeloma patients (PBLs and MILs), as would be the case when employing bispecific antibodies in a myeloma patient, or with activated PBLs and MILs. For each challenge (every two day), OPM-2 myeloma tumor cells were added to the PBLs or MILs co-cultured with the BCMA bispecific antibody, and the percent of cell lysis (i.e., the tumor lysis activity of the MILs or PBLs) was evaluated. As shown in FIG. 2, there was no tumor killing with inactivated MILs (the T-cells corresponding to the T-cells the bispecific that would naturally engage considering that myeloma is a disease of the bone marrow). PBLs demonstrated marginal activity early which then disappeared upon re-stimulation. In sharp contrast, T-cell activation significantly increased the anti-tumor efficacy of the bispecific. Both aMILs and aPBLs T-cell were found capable of increasing anti-tumor efficacy, but it was significantly greater with activated MILs especially upon repetitive tumor rechallenges.

Specifically, FIG. 2 shown that activated T-cells increased the efficacy of BCMA bispecific antibodies by increasing the percentage of OPM-2 myeloma tumor cell lysis upon incubation with a BCMA bispecific antibody and T-cells. Tumor was added every two days and percent lysis was evaluated.

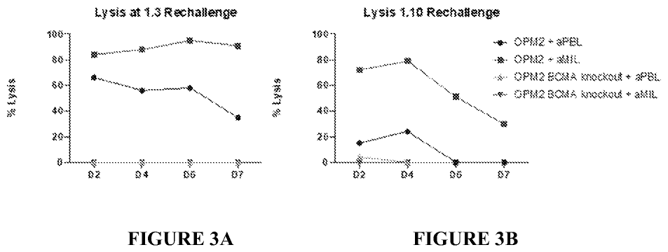

Considering the superior efficacy of aMILs in enhancing the efficacy of these bispecific antibodies, the effect of a lower effector to target ratio was determined (FIG. 3). As illustrated in FIGS. 3A and 3B, it was shown that at a 1:10 ratio, the anti-tumor effect of MILs was even more pronounced compared to PBLs and preserved its function upon tumor rechallenge. aMILs shown a greater anti-tumor efficacy compared to aPBLs that persisted upon rechallenge.

### Example 2

### BCMA-Specific Bispecific Antibody

Preparation of T Cells: Bone marrow and blood were collected from multiple myeloma (MM) patients. Bone marrow mononuclear cells (BM) and peripheral blood mononuclear cells (PBL) were isolated with a lymphocyte separation media (LSM, LONZA) and frozen. For the cells that underwent expansion, cryopreserved cells were thawed and stimulated with CD3/CD28 beads (Life Technologies) at a predefined bead to T-cell ratio and recombinant human IL-2 (200 IU/ml). Cells were grown for 7-10 days. CD3/CD28 beads were removed from cultures using a magnet (Miltenyi) and CD3% was determined by flow cytometry. The cells were then viably frozen after their expansion. The unactivated CD3+ T cells were isolated from the BM/PBL using Miltenyi Pan T cell isolation Kit (#130-096-535) and are referred to as uMIL and uPBL, respectively. Less than 24 hours before the assay, unactivated (u) and activated (a) PBLs and MILs were thawed and rested in AIM-V media overnight in incubators with 37° C. in a 5% CO₂.

Cell lines and cell culture: All tumor cell lines were of human origin and obtained from the ATCC. OPM-2 G-L (GFP-Luciferase) and OPM-2 G-L BCMA knockout (OPM-2 and OPM-2 BCMA KO, respectively) were obtained and cultured in RPMI 1640 medium with 10% fetal bovine serum with antibiotics at 37° C. in a 5% CO₂.

T Cell-Tumor Co-Culture: Activated MILs/PBL and unactivated MIL/PBL were plated in a 96-well round-bottom plate together with MM target cells for a final E:T ratio of 1:10. 100 ul of BCMA bispecific antibody was added for a final concentration of 10⁻⁸ M. Every 48 hours (day 2, 4, 6, and 8) the samples were re-challenged with tumor and bispecific antibody. At the same concentration as prior.

Flow cytometry analysis: Samples were harvested on days 2, 4, 6, and 8. Viability was determined using Zombie NIR (Biolegend). Tumor cells were identified by CD138 expression. Myeloma-specific cytotoxicity was calculated with the following formula: % Lysis=100−(viable cells of the treatment group×100/viable cells of the control group). T cell activation was measured by the expression of 4-1BB and OX40. Polyfunctionality of T cells was determined by co-expression of CD107a, TNFa, and IFNg. T cell exhaustion was measured by the expression of PD-1 and TIGIT. CD3 Fold expansion was determined by calculating the number of cells at each time point and dividing them by the number of CD3+ cells at Day 0. Samples were analyzed on a Cytek Northern Lights and processed with FlowJo software (V10, Tree Star) and Graphpad Prism.

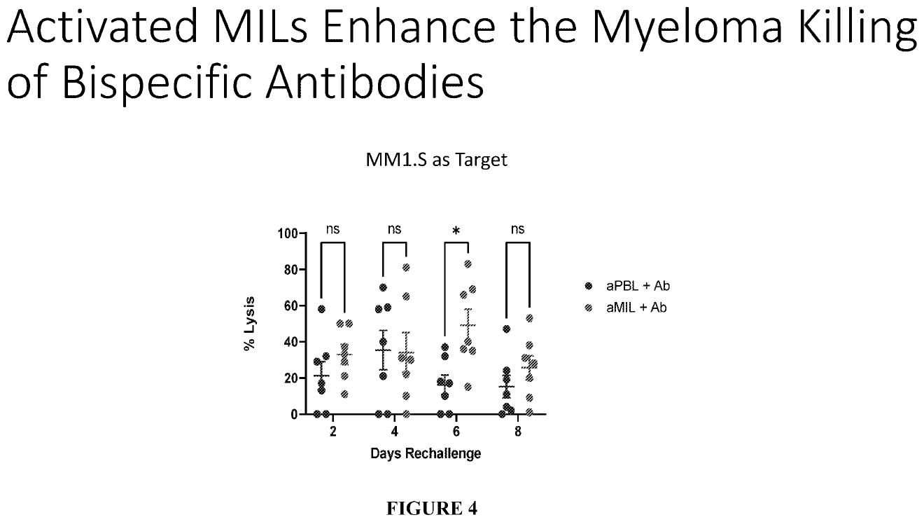

Activated PBLs or activated MILs were added to a BCMA-specific bispecific antibody and % lysis of the myeloma cell line, MIM1.S, was determined serially over 8 days as indicated (FIG. 4). As illustrated in FIG. 4, it was shown that activated MILs enhance the myeloma killing of bispecific antibodies.

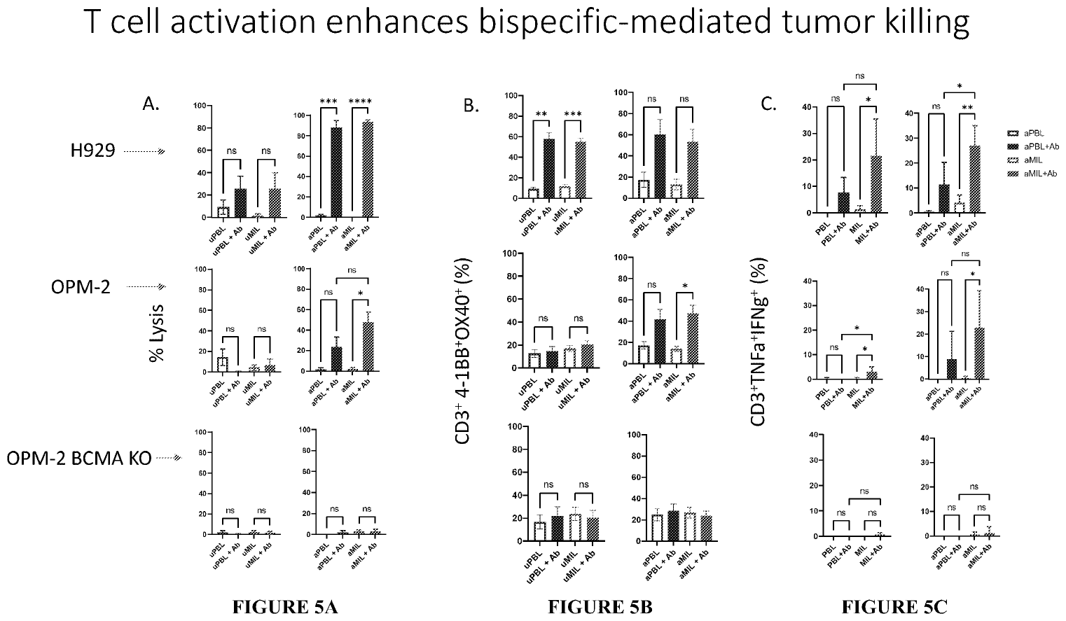

Myeloma cell killing was determined by co-culturing a BCMA-specific bispecific antibody with either activated or unactivated PBLs or MILs as indicated in FIG. 5. The myeloma lines that were used were H929 (a high BCMA expressing line) and OPM-2 (low BCMA expressing) or the OPM-2 BCMA knock-out line. Cell killing as determined by percent lysis (FIG. 5A), T cell activation as determined by 4-1BB and OX40 expression (FIG. 5B), as well the polyfunctionality as determined by TNF-alpha and interferon-gamma expression (FIG. 5C) were determine for each condition. As illustrated in FIG. 5, overall, T cell activation appeared to significantly enhance the potency of the BCMA-specific bispecific antibody. Activated MILs appeared to show even greater benefit when the level of BCMA expression is lower (OPM-2 vs H929) (FIG. 5).

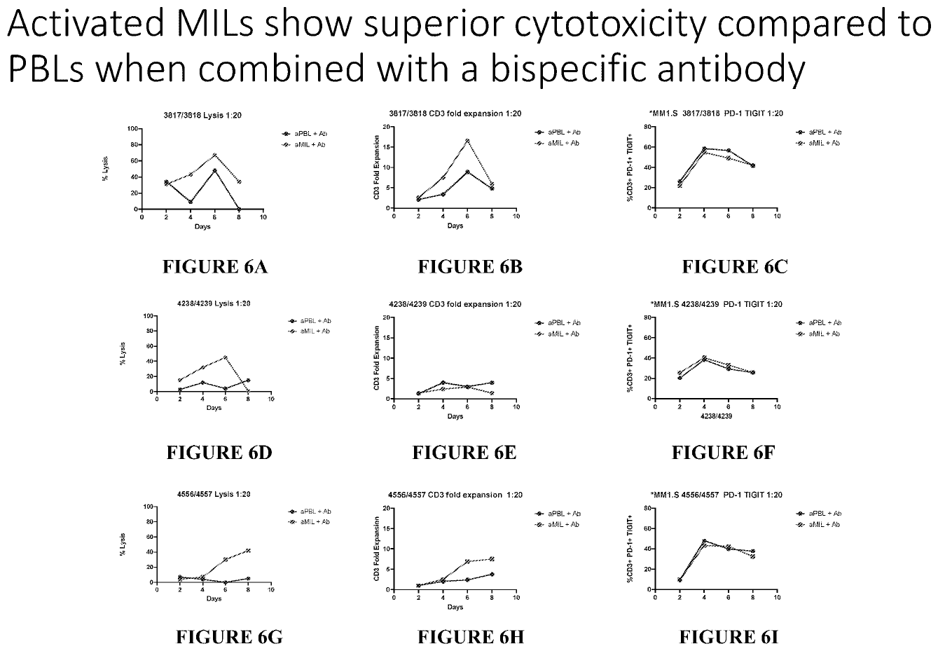

As illustrated in FIG. 6, activated MILs showed superior cytotoxicity compared to PBLs when combined with BCMA-specific bispecific antibody.

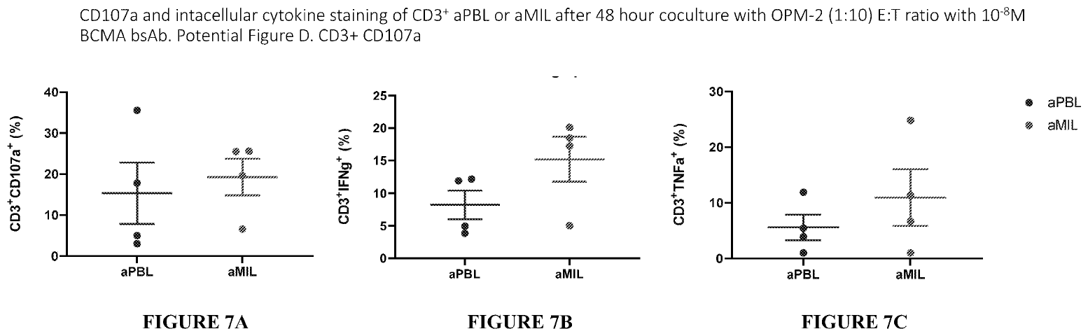

FIG. 7 shows CD107a and intracellular cytokine (INFg and TNFa) staining of CD3+ aPBL or aMIL after 48 hour coculture with OPM-2 (1:10) E:T ratio with 10⁻⁸ M BCMA-specific bispecific antibody. Although not statistically significant, a trend of greater killing (CD107a) expression (FIG. 7A), greater IFNg production (FIG. 7B), and greater TNFa production (FIG. 7C), was illustrated in patients.

Although the invention has been described with reference to the above examples, it will be understood that modifications and variations are encompassed within the spirit and scope of the invention. Accordingly, the invention is limited only by the following claims.

What is claimed is:

1. A method of treating cancer in a subject comprising administering to the subject an activated T-cell and a bispecific T-cell engager (BiTE) or a bispecific antibody, thereby treating cancer in the subject.

2. The method of claim 1, wherein the activated T-cell is an activated marrow infiltrating lymphocyte (aMIL) or an activated peripheral blood lymphocyte (aPBL).

3. The method of claim 1, wherein activating the T-cell is ex vivo.

4. The method of claim 3, wherein activating a T-cell comprises co-stimulating CD3 and CD28.

5. The method of claim 4, wherein co-stimulating CD3 and CD28 comprises contacting the T-cell with an anti-CD3 antibody and an anti-CD28 antibody.

6. The method of claim 3, wherein activating a T-cell comprises enhancing anti-tumoral activity, restoring tumor-mediated immunity and/or reversing T-cell tolerance to tumor-specific antigen.

7. The method of claim 6, wherein activating a T-cell comprises increasing T-cell-mediated tumor cell lysis.

8. The method of claim 1, wherein the bispecific antibody targets a tumor antigen and a T-cell.

9. The method of claim 8, wherein the bispecific antibody is an anti-B-cell maturation antigen (BCMA)-bispecific antibody.

10. The method of claim 1, wherein the activated T-cell is contacted with the BiTEs or with the bispecific antibody prior to administration to the subject.

11. The method of claim 1, wherein the activated T-cell has an increased expression of OX40 and/or 4-IBB.

12. The method of claim 1, wherein the subject has a hematologic cancer or a solid tumor.

13. The method of claim 1, further comprising administering to the subject an anti-cancer treatment.

14. A method of enhancing an anti-tumoral activity of a bispecific T-cell engager (BiTE) or of a bispecific antibody comprising contacting the BiTE or the bispecific antibody with an activated T-cell.

15. The method of claim 14, wherein the activated T-cell has a restored T-cell functionality.

16. The method of claim 15, wherein the T-cell has increased T-cell mediated cytotoxicity.

17. The method of claim 14, wherein enhancing anti-tumoral activity comprises restoring tumor-mediated immunity and/or reversing T-cell tolerance to tumor-specific antigen.

18. The method of claim 14, further comprising activating a T-cell.

19. The method of claim 18, wherein activating a T-cell comprises co-stimulating CD3 and CD28.

20. The method of claim 19, wherein co-stimulating CD3 and CD28 comprises contacting the T-cells with an anti-CD3 antibody and an anti-CD28 antibody.
